(19) United States
(12) Patent Application Publication
(10) Pub. No.: US 2024/0293302 A1
(43) Pub. Date: Sep. 5, 2024

(54) Solid Personal Care Compositions and Methods for Preventing and Treating Pollution Damage to Skin

(71) Applicant: Colgate-Palmolive Company, New York, NY (US)

(72) Inventors: Jesus Ivan ROMERO, Mexico City (MX); Reina ESPINOSA, Mexico City (MX); Sara MORALES, Mexico City (MX); Aaron COHEN, New Brunswick, NJ (US); Junhong MAO, Plainsboro, NJ (US); Aixing FAN, Bridgewater, NJ (US)

(73) Assignee: Colgate-Palmolive Company, New York, NY (US)

(21) Appl. No.: 18/547,950

(22) PCT Filed: Feb. 23, 2022
(86) PCT No.: PCT/US2022/017421

§ 371 Date: Aug. 25, 2023

Related U.S. Application Data
(60) Provisional application No. 63/155,025, filed on Mar. 1, 2021.

(51) Int. Cl. A61K 8/67; A61K 8/02; A61Q 17/00; A61Q 19/10

(52) U.S. Cl. CPC A61K 8/678; A61K 8/0216; A61K 8/676; A61Q 17/00; A61Q 19/10

(57) ABSTRACT

Personal care compositions, such as solid personal care compositions, and methods for preventing and/or treating skin pollution or lipid peroxidation of skin are disclosed. The personal care compositions may include a carrier and an antioxidant system. The antioxidant system may include one or more antioxidants and may be present in an effective amount to treat, inhibit, or prevent a lipid peroxidation of skin.

### CROSS-REFERENCE TO RELATED APPLICATION

This application claims the benefit of priority from U.S. Provisional Application No. 63/155,025, filed Mar. 1, 2021, the contents of which are hereby incorporated herein by reference in their entirety.

### BACKGROUND

Signs of aging often appear on skin as fine lines and wrinkles, age spots, dryness, blotchy discolorations, and sagging. Similarly, signs of aging may also manifest in hair as frizziness, dullness, and hair loss. These signs of aging or damage to the skin and hair are often exacerbated by other factors, such as environmental or extrinsic factors. For example, signs of aging or damage to the skin and hair may often be accelerated by exposure to environmental pollution, ultraviolet radiation, ozone, free radicals, or the like. Air pollution in particular includes microparticles that causes the production of free radical and reactive oxygen species (ROS) in skin, thereby resulting in skin damage via lipid peroxidation. While antioxidants are often utilized to prevent or inhibit cell damage, effectively delivering the antioxidants remains a challenge.

What is needed, then, are personal care compositions and methods for preventing and/or treating pollution damage to skin.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a personal care composition including a carrier and an antioxidant system having one or more antioxidants. The antioxidant system may be present in an effective amount to treat, inhibit, or prevent lipid peroxidation of skin.

In at least one implementation, the one or more antioxidants may include one or more of selenium, carotenoids, lycopene, lutein, zeaxanthin, one or more vitamins, or combinations thereof.

In at least one implementation, the one or more antioxidants may include one or more vitamins. The one or more vitamins may include vitamin C, vitamin E, or combinations thereof. The one or more vitamins may include a combination of vitamin C and vitamin E. The vitamin E may include one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof. The vitamin E may include gamma-tocopherol. The vitamin C may include ascorbic acid, derivatives thereof, or combinations thereof. The derivatives of ascorbic acid may include one or more of L-ascorbic acid, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof. The derivatives of ascorbic acid may include sodium ascorbyl phosphate. In an exemplary implementation, the vitamin C may include sodium ascorbyl phosphate, and the vitamin E may include gamma-tocopherol.

In at least one implementation, the carrier may include a cleansing component, and wherein the cleansing component may include a soap. The soap may include a fatty acid soap. The soap may include C₁₆-C₁₈ fatty acids and C₁₂-C₁₄ fatty acids. The C₁₆-C₁₈ fatty acids may be present in an amount of from about 85 wt % to about 95 wt %, and the C₁₂-C₁₄ fatty acids may be present in an amount of from about 5 wt % to about 15 wt %, based on a total weight of the soap. In one implementation, the C₁₆-C₁₈ fatty acids may be present in an amount of about 92.5 wt %, and the C₁₂-C₁₄ fatty acids may be present in an amount of about 7.5 wt %, based on a total weight of the soap

In at least one implementation, the personal care composition may be a solid personal care composition.

The foregoing and/or other aspects and utilities embodied in the present disclosure may be achieved by providing a method for preparing the personal care composition of any one of the foregoing claims for treating or preventing lipid peroxidation of skin, the method comprising contacting the carrier and the antioxidant system with one another.

The foregoing and/or other aspects and utilities embodied in the present disclosure may be achieved by providing a method for preventing lipid peroxidation of skin. The method may include contacting any one or more of the personal care compositions disclosed herein with the skin, and exposing the skin to pollution. The personal care composition may prevent lipid peroxidation of the skin.

The foregoing and/or other aspects and utilities embodied in the present disclosure may be achieved by providing a method for treating lipid peroxidation of skin. The method may include contacting any one or more of the personal care compositions disclosed herein with the skin. Contacting the personal care composition with the skin may reduce lipid peroxidation in and on the skin.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that personal care compositions, such as solid personal care compositions (e.g., solid cleansing compositions), including a carrier and one or more antioxidants, prevent pollution damage or lipid peroxidation in and/or on skin when applied to the skin prior to exposure to ozone. Particularly, the inventors have surprisingly and unexpectedly discovered that personal care compositions, including a carrier and a combination of vitamin E and vitamin C, prevent pollution damage or lipid peroxidation in and/or on skin when applied to the skin prior to exposure to ozone. The vitamin E may be or include gamma-Tocopherol (γ-Tocopherol). The vitamin C may be or include ascorbic acid, a derivative of ascorbic acid, or combinations thereof. The vitamin E and the vitamin C may be present in the personal care composition in a weight ratio of from about 1:1 to about 60:1, about 20:1 to about 40:1, or about 30:1. The one or more antioxidants may be present in the personal care composition in an amount of from about 0.005 wt % to about 2 wt %.

The present inventors have also surprisingly and unexpectedly discovered that personal care compositions, such as solid personal care compositions, including a carrier and one or more antioxidants reduces, removes, or otherwise treats pollution damage or lipid peroxidation in and/or on skin when applied to the skin prior to exposure to ozone. Particularly, the inventors have surprisingly and unexpectedly discovered that personal care compositions, including a carrier and a combination of vitamin E and vitamin C, reduce, remove, or otherwise treat pollution damage or lipid peroxidation in and/or on skin when applied to the skin prior to exposure to ozone. The vitamin E may be or include gamma-Tocopherol (γ-Tocopherol). The vitamin C may be or include ascorbic acid, a derivative thereof, such as sodium ascorbyl phosphate, or combinations thereof. The vitamin E and the vitamin C may be present in the personal care composition in a weight ratio of from about 1:1 to about 60:1, about 20:1 to about 40:1, or about 30:1. The one or more antioxidants may be present in the personal care composition in an amount of from about 0.005 wt % to about 2 wt %.

### Compositions

Compositions disclosed herein may be or include a personal care product or a personal care composition thereof. For example, compositions disclosed herein may be a personal care composition, a personal care product, or form a portion of the personal care composition or the personal care product. In an exemplary implementation, the compositions disclosed herein are personal care composition including a carrier or vehicle and an effective amount of one or more antioxidants or an antioxidant system including the one or more antioxidants. The carrier or vehicle may be capable of or configured to store, entrain, or otherwise contain the antioxidant system and/or the one or more antioxidants thereof, and deliver the antioxidant system and/or the one or more antioxidants thereof to one or more tissues, such as skin. The antioxidant system and/or the one or more antioxidants thereof may be capable of or configured to prevent pollution damage or lipid peroxidation in and/or on skin when applied to the skin prior to exposure to ozone. The antioxidant system and/or the one or more antioxidants thereof may also be capable of or configured to reduce, remove, or treat pollution damage or lipid peroxidation in and/or on skin when applied to the skin after exposure to ozone.

Illustrative personal care products or compositions thereof that may include the carrier and the antioxidant system and/or the one or more antioxidants thereof may include, but are not limited to, bar soaps, body washes, shower gels, liquid soaps, face washes, shampoos, hair conditioners, lotions, moisturizers, serums, solid personal care compositions, such as solid soaps, or the like. In at least one implementation, the personal care compositions disclosed herein may be or include solid personal care compositions, such as solid cleansing compositions. Illustrative solid cleansing compositions may be or include, but are not limited to, bar soap compositions, cleansing bars, and/or other solid cleansing compositions that may be used for personal cleansing or as a laundry bar. In a preferred implementation, the personal care product or the composition thereof that includes the carrier and the surfactant system are solid cleansing composition, such as bar soaps.

### Antioxidant and Antioxidant System

The personal care composition may include one or more antioxidants or an antioxidant system including the one or more antioxidants. Illustrative antioxidants may be or include, but are not limited to, selenium, carotenoids, lycopene, lutein, zeaxanthin, one or more vitamins, or combinations thereof. In a preferred implementation, the one or more antioxidants include the one or more vitamins. The one or more vitamins may be or include but are not limited to, beta-carotene, vitamin C, vitamin E, or combinations thereof. In at least one implementation, the one or more antioxidants include vitamin C, vitamin E, or combinations thereof, and preferably a combination of vitamin C and vitamin E.

Generally, vitamin E may refer to a generic name for a family of four isomers of tocopherols and four isomers of tocotrienols. All eight isomers of vitamin E have a 6-chromanol ring structure and a side chain. The four tocopherols include fully saturated side chains and include alpha-tocopherol, gamma-tocopherol, beta-tocopherol, and delta-tocopherol. The four tocotrienols include unsaturated side chains and include alpha-tocotrienol, gamma-tocotrienol, beta-tocotrienol, and delta-tocotrienol. As used herein, the term or expression “vitamin E” may refer to any one or more of the eight isomers. For example, as used herein, vitamin E may be or include one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof. In at least one implementation, the vitamin E includes at least one of the four tocopherols. In a preferred implementation, the vitamin E includes gamma-tocopherol, and gamma-tocopherol may make up the major component of the vitamin E. For example, the vitamin E may include gamma-tocopherol in an amount relatively greater than any one or more of the other isomers of vitamin E. In at least one implementation, the vitamin E includes only gamma-tocopherol or includes substantially only gamma-tocopherol. It should be appreciated that the vitamin E and/or the isomers thereof may be or include natural forms of vitamin E, synthetic forms of vitamin E, or combinations thereof. Any one or more of the isomers of vitamin E may be in the “d” form, the “1” form, or combinations thereof. In a preferred implementation, the “d” form is present. For example, the vitamin E may include d-gamma-tocopherol.

As discussed above, the vitamin C may be ascorbic acid or derivatives thereof. Ascorbic acid exists as two enantiomers commonly denoted “l” (for “levo”) and “d” (for “dextro”). The “l” isomer is the one most often encountered. Ascorbic acid is also referred to as L(+)-ascorbic acid or l-ascorbic acid. The ascorbic acid derivatives may be or include, but are not limited to, L-ascorbic acid, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-l-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof. In a preferred implementation, the vitamin C comprises sodium ascorbyl phosphate.

The personal care composition may include the antioxidant system and/or any one or more of the antioxidants thereof in an amount of from about 0.005 wt % to about 2 wt %, by total weight of the personal care composition. For example, the antioxidant system and/or any one or more of the antioxidants thereof may be present in an amount of greater than or equal to 0.005 wt %, greater than or equal to 0.01 wt %, greater than or equal to 0.02 wt %, greater than or equal to 0.03 wt %, greater than or equal to 0.04 wt %, greater than or equal to 0.05 wt %, greater than or equal to 0.06 wt %, greater than or equal to 0.07 wt %, greater than or equal to 0.08 wt %, greater than or equal to 0.09 wt %, greater than or equal to 0.1 wt %, greater than or equal to 0.2 wt %, greater than or equal to 0.3 wt %, greater than or equal to 0.4 wt %, greater than or equal to 0.5 wt %, greater than or equal to 0.6 wt %, greater than or equal to 0.7 wt %, greater than or equal to 0.8 wt %, greater than or equal to 0.9 wt %, greater than or equal to 0.95 wt %, greater than or equal to 1 wt %, greater than or equal to 1.1 wt %, greater than or equal to 1.2 wt %, greater than or equal to 1.3 wt %, greater than or equal to 1.4 wt %, greater than or equal to 1.5 wt %, greater than or equal to 1.6 wt %, greater than or equal to 1.7 wt %, greater than or equal to 1.8 wt %, or greater than or equal to 1.9 wt %, or up to 2 wt %, based on the total weight of the personal care composition. In another example, the antioxidant system and/or any one or more of the antioxidants thereof may be present in an amount of less than or equal to 2 wt %, less than or equal to 1.9 wt %, less than or equal to 1.8 wt %, less than or equal to 1.6 wt %, less than or equal to 1.4 wt %, less than or equal to 1.2 wt %, less than or equal to 0.95 wt %, less than or equal to 0.9 wt %, less than or equal to 0.8 wt %, less than or equal to 0.7 wt %, less than or equal to 0.6 wt %, less than or equal to 0.5 wt %, less than or equal to 0.4 wt %, less than or equal to 0.3 wt %, less than or equal to 0.2 wt %, less than or equal to 0.1 wt %, less than or equal to 0.09 wt %, less than or equal to 0.08 wt %, less than or equal to 0.07 wt %, less than or equal to 0.06 wt %, less than or equal to 0.05 wt %, less than or equal to 0.04 wt %, less than or equal to 0.03 wt %, or less than or equal to 0.02 wt %, based on the total weight of the personal care composition. In a preferred example, the personal care composition may include the antioxidant system in an amount of from about 0.01 wt % to about 0.5 wt %, more preferably in an amount of from about 0.2 wt % to about 0.4 wt %.

As discussed above, the antioxidant system may include a combination of vitamin E and vitamin C. In at least one implementation, the vitamin E and the vitamin C may be present in the personal care composition or the antioxidant system thereof in a weight ratio of from about 1:1 to about 60:1. For example, the vitamin E and the vitamin C may be present in the personal care composition or the antioxidant system thereof in a weight ratio of from about 1:1 to about 60:1, about 10:1 to about 50:1, about 20:1 to about 40:1, more preferably about 25:1 to about 35:1, even more preferably about 30:1.

In at least one implementation, the one or more antioxidants of the antioxidant system include sodium ascorbyl phosphate, gamma-tocopherol, or combinations thereof. In a preferred implementation, the one or more antioxidants include a combination of sodium ascorbyl phosphate and gamma-tocopherol.

The personal care composition or the antioxidant system thereof may include vitamin E in an amount of from about 0.01 wt % to about 2 wt %, based on the total weight of the personal care composition or the antioxidant system thereof. For example, the vitamin E may be present in an amount of greater than or equal to 0.01 wt %, greater than or equal to 0.02 wt %, greater than or equal to 0.03 wt %, greater than or equal to 0.04 wt %, greater than or equal to 0.05 wt %, greater than or equal to 0.06 wt %, greater than or equal to 0.07 wt %, greater than or equal to 0.08 wt %, greater than or equal to 0.09 wt %, greater than or equal to 0.1 wt %, greater than or equal to 0.15 wt %, greater than or equal to 0.2 wt %, greater than or equal to 0.25 wt %, greater than or equal to 0.3 wt %, greater than or equal to 0.35 wt %, greater than or equal to 0.4 wt %, greater than or equal to 0.45 wt %, greater than or equal to 0.5 wt %, greater than or equal to 0.55 wt %, greater than or equal to 0.8 wt %, greater than or equal to 1 wt %, greater than or equal to 1.2 wt %, greater than or equal to 1.6 wt %, greater than or equal to 1.8 wt %, or more, based on the total weight of the personal care composition. In another example, the vitamin E may be present in an amount of less than or equal to 2 wt %, less than or equal to 1.8 wt %, less than or equal to 1.4 wt %, less than or equal to 1.2 wt %, less than or equal to 1 wt %, less than or equal to 0.95 wt %, less than or equal to 0.9 wt %, less than or equal to 0.8 wt %, less than or equal to 0.7 wt %, less than or equal to 0.6 wt %, less than or equal to 0.55 wt %, less than or equal to 0.5 wt %, less than or equal to 0.45 wt %, less than or equal to 0.4 wt %, less than or equal to 0.35 wt %, less than or equal to 0.3 wt %, less than or equal to 0.25 wt %, less than or equal to 0.2 wt %, or less than or equal to 0.15 wt %, based on the total weight of the personal care composition. In a preferred example, the vitamin E may be present in an amount of from about 0.1 wt % to about 0.5 wt %, about 0.2 wt % to about 0.4 wt %, or about 0.3 wt %, based on a total weight of the personal care composition.

The personal care composition or the antioxidant system thereof may include vitamin C in an amount of from about 0.005 wt % to about 2 wt %, based on the total weight of the personal care composition or the antioxidant system thereof. For example, the vitamin C may be present in an amount of greater than or equal to 0.005 wt %, greater than or equal to 0.006 wt %, greater than or equal to 0.007 wt %, greater than or equal to 0.008 wt %, greater than or equal to 0.009 wt %, greater than or equal to 0.01 wt %, greater than or equal to 0.02 wt %, greater than or equal to 0.03 wt %, greater than or equal to 0.04 wt %, greater than or equal to 0.05 wt %, greater than or equal to 0.06 wt %, greater than or equal to 0.07 wt %, greater than or equal to 0.08 wt %, greater than or equal to 0.09 wt %, greater than or equal to 0.1 wt %, greater than or equal to 0.5 wt %, greater than or equal to 1 wt %, greater than or equal to 1.5 wt %, greater than or equal to 1.8 wt %, or more, based on the total weight of the personal care composition. In another example, the vitamin C may be present in an amount of less than or equal to 2 wt %, less than or equal to 1.8 wt %, less than or equal to 1.6 wt %, less than or equal to 1.4 wt %, less than or equal to 1.2 wt %, less than or equal to 1 wt %, less than or equal to 0.9 wt %, less than or equal to 0.8 wt %, less than or equal to 0.7 wt %, less than or equal to 0.6 wt %, less than or equal to 0.5 wt %, less than or equal to 0.4 wt %, less than or equal to 0.3 wt %, less than or equal to 0.2 wt %, less than or equal to 0.1 wt %, less than or equal to 0.05 wt %, less than or equal to 0.04 wt %, less than or equal to 0.03 wt %, less than or equal to 0.02 wt %, less than or equal to 0.01 wt %, or less, based on the total weight of the personal care composition. In a preferred example, the vitamin C may be present in an amount of from about 0.005 wt % to about 0.1 wt %, about 0.008 wt % to about 0.05 wt %, or about 0.01 wt %.

### Carrier or Vehicle

The carrier of the personal care composition (e.g., solid cleansing composition) may include at least one cleansing component or agent. In at least one implementation, the cleansing component may include or refer to a soap and/or surfactant. For example, the cleansing component may be or include one or more soaps, one or more surfactants, or combinations thereof. The amount of the cleansing component present in the personal care composition may be from about 85 wt % to about 99 wt %, based on the total weight of the personal care composition. For example, the cleaning component may be present in an amount of from greater than or equal to 85 wt %, greater than or equal to 90 wt %, greater than or equal to 92 wt %, greater than or equal to 94 wt %, greater than or equal to 96 wt %, greater than or equal to 97 wt %, or greater than or equal to 97.5 wt % to about 99 wt %, based on the total weight of the personal care composition. In another example, the cleaning component may be present in an amount of from about 95 wt % to about 99 wt %, about 96 wt % to about 98 wt %, or about 97 wt %, based on the total weight of the personal care composition.

The soap may be or include alkali metal or alkanol ammonium salts of aliphatic alkane- or alkene-monocarboxylic acids, including about 6 to about 22 carbon atoms, about 8 to about 22 carbon atoms, about 6 to about 18 carbon atoms, or about 12 to about 18 carbon atoms. Illustrative soaps that may be utilized in the solid cleansing composition may be or include, but are not limited to, sodium soaps, ammonium soaps, potassium soaps, magnesium soaps, calcium soaps, and the like, or mixtures thereof. In a preferred implementation, the base component or soap includes a sodium soap; however, it should be appreciated that at least a portion of the soap may also include one or more ammonium soaps, potassium soaps, magnesium soaps, calcium soaps, and the like, or mixtures and combinations thereof. In a preferred implementation, the base component or the soap may be or include, but is not limited to, alkali metal salts of aliphatic (alkanoic or alkenoic) acids having about 8 to about 22 carbon atoms or about 10 to about 20 carbon atoms.

The soap may be a fatty acid soap. The fatty acid soap may include one or more neutralized fatty acids. Illustrative fatty acids used for the fatty acid soap may be or include, but are not limited to, myristic acid, lauric acid, palmitic acid, oleic acid, stearic acids, or the like, or combinations thereof. Sources of fatty acids may include coconut oil, palm oil, palm kernel oil, tallow, avocado, canola, corn, cottonseed, olive, hi-oleic sunflower, mid-oleic sunflower, sunflower, palm stearin, palm kernel olein, safflower, and babassu oils.

The fatty acids may be neutralized with any base to form the soap or fatty acid soap. Illustrative bases may be or include, but are not limited to, sodium hydroxide, potassium hydroxide, triethanolamine, and the like, or mixtures and combinations thereof. In certain implementations, the fatty acid soap may be formed from fatty acids neutralized by two or more bases. In certain embodiments, the bases are sodium hydroxide and triethanolamine. In certain implementations, the molar ratio of sodium hydroxide and triethanolamine is 1:1. In certain implementations, the fatty acids may be or include any one or more of oleic acid, palmitic acid, stearic acid, and lauric acid. For example, the fatty acid soap may be or include sodium palmitate, sodium oleate, sodium laurate, sodium stearate, or any combination or mixture thereof. In at least one implementation, the fatty acid soap may further include glycerin.

In at least one implementation, the soap may refer to the salts of fatty acids that may typically be used to make soap bars. For example, soap may be or include a mixture or blend of about 85 wt % to about 95 wt % C₁₆-C₁₈ fatty acids and about 5 wt % to about 15 wt % C₁₂-C₁₄ fatty acids, based on the total weight of the soap. The C₁₆-C₁₈ fatty acids may be obtained from tallow and the C₁₂-C₁₄ fatty acids may be obtained from lauric, palm kernel, or coconut oils. In at least one implementation, the soap may include about 85 wt % to about 95 wt % tallow, about 5 wt % to about 15 wt % lauric acid, optionally about 3 wt % to about 7 wt % syndet, or combinations thereof. In a preferred implementation, the soap may include about 90 wt % to about 95 wt % or about 92.5 wt % tallow, about 5 wt % to about 10 wt % or about 7.5 wt % lauric acid, and optionally about 3 wt % to about 7 wt % or about 5 wt % syndet.

The amount or concentration of the soap in the personal care composition or the carrier thereof may vary widely. In at least one implementation, the amount of the soap in the personal care composition or the carrier thereof may be greater than or equal to 50 weight % and less than or equal to 99 weight %. For example, the soap or cleansing component may be present in the personal care composition or the carrier thereof in an amount of from about 50 wt %, about 80 wt %, about 90 wt %, about 95 wt %, 96 wt %, about 97 wt %, or about 98 wt % to about 99 wt %, based on the total weight of the personal care composition of the carrier thereof. In a preferred implementation, the soap or cleansing component may be present in the personal care composition or the carrier thereof in an amount of from about 97 wt % to about 99 wt %, more preferably about 98 wt %, based on the total weight of the personal care composition of the carrier thereof.

The soap of the cleansing component may be or include one or more surfactants. For example, the soap may include one or more anionic surfactants, one or more amphoteric surfactants, one or more cationic surfactants, one or more zwitterionic surfactants, one or more nonionic surfactants, and mixtures thereof. Examples of suitable surfactants may be found in U.S. Pat. No. 3,959,458 to Agricola et al., U.S. Pat. No. 3,937,807 to Haefele, and U.S. Pat. No. 4,051,234 to Gieske et al, the disclosures of which are incorporated herein by reference to the extent consistent with the present disclosure. Any other surfactant may also be present in the soap including, but not limited to, sulfate, sulfonate alpha olefin sulfonates, isethionates such as SCI, N-alkyl or N-acyl taurates, sulfosuccinate, phosphates, glycinates, amphoteric surfactants, such as betaines, sulfobetaines and the like, and nonionic surfactants, such as alkanolamide, alkylpolyglycosides.

The personal care composition and/or the carrier thereof may include water. Water of the personal care composition may be deionized water, demineralized water, and/or softened water. Water of the personal care composition and/or the carrier thereof may be separate from the water of other components of the personal care composition and/or the carrier thereof. For example, water of the soap may be separate from water in one or more components of the personal care composition. Water may make up the balance of the personal care composition. For example, the amount of water in the personal care composition may be from greater than 0 weight % to about 10 weight %, about 10 weight % to about 20 weight %, about 12 weight % to about 18 weight %, or about 14 weight % to about 16 weight %. In another example, the amount of water in the personal care composition may be from greater than 0 weight %, at least 10 weight %, at least 11 weight %, at least 12 weight %, at least 13 weight %, at least 14 weight %, at least 15 weight %, at least 16 weight %, or at least 17 weight %. In at least one implementation, the amount of water may be about 0.1 wt %, about 0.5 wt %, about 1 wt %, about 10 weight %, about 11 weight %, about 12 weight %, about 13 weight %, about 14 weight %, or about 15 weight %. The amount of water in the personal care composition may include free water added and water introduced with other components or materials of the personal care composition. For example, the amount of water in the personal care composition may include free water and water associated with the soap, the antioxidant system, and/or any other component of the personal care composition.

The personal care composition and/or the carrier thereof may include charcoal. The charcoal may be in the form of particles, such as beads, spheres, microparticles, agglomerations, or the like, or combination thereof. The charcoal may be activated charcoal. The charcoal may be present in the personal care composition and/or the carrier thereof in an amount of from greater than 0 wt % to about 0.2 wt %, based on the total weight of the personal care composition and/or the carrier thereof. For example, the charcoal may be present in an amount of from greater than 0 wt %, about 0.02 wt %, about 0.04 wt %, about 0.06 wt %, about 0.08 wt %, or about 0.09 wt % to about 0.1 wt %, about 0.12 wt %, about 0.14 wt %, about 0.16 wt %, about 0.18 wt %, or about 0.2 wt %, based on the total weight of the personal care composition and/or the carrier thereof. In a preferred implementation, the charcoal may be present in an amount of from about 0.06 wt % to about 0.14 wt %, more preferably about 0.08 wt % to about 0.12 wt %, or about 0.1 wt %, based on the total weight of the personal care composition and/or the carrier thereof.

The personal care composition may include one or more fragrances. The one or more fragrances may be present in the personal care composition in an amount of from greater than 0 wt % to about 5 wt %, based on the total weight of the personal care composition. For example, the one or more fragrances may be present in an amount of from greater than 0 wt %, about 0.5 wt %, about 1 wt %, about 1.5 wt %, or about 2 wt % to about 2.5 wt %, about 3 wt %, about 3.5 wt %, about 4 wt %, about 4.5 wt %, or about 5 wt %, based on the total weight of the personal care composition. In a preferred implementation, the one or more fragrances may be present in an amount of from about 1 wt % to about 3 wt %, more preferably about 1.5 wt % to about 2.5 wt %, or about 2 wt %, based on the total weight of the personal care composition.

### Humectants

The personal care composition may include one or more humectants. Illustrative humectants may include, but are not limited to, ascorbyl dipalmitate, acetamide MEA, glucose glutamate, glucuronic acid, TEA-lactate, TEA-PCA, corn syrup, fructose, glucose, glycerin, glycol, 1,2,6-hexanetriol, sodium lactate, sodium PCA, hydrogenated starch hydrolysate, inositol, lactic acid, lactose, mannitol, PCA, PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, glutamic acid, glycereth-7, glycereth-12, glycereth-26, saccharide isomerate, sorbeth-20, sorbitol, sucrose, thioglycerin, tris-(hydroxymethyl)nitromethane, tromethamine, histidine, PEG-75, PEG-135, PEG-150, PEG-200, PEG-5 pentaerythritol ether, polyglyceryl sorbitol, sorbitol, urea, xylitol, or the like, or combinations thereof.

### Free Fatty Acids

In at least one implementation, the personal care composition may include one or more free fatty acids configured to provide enhanced skin feel benefits. For example, the personal care composition may include the fatty acids to provide softer or smoother feeling skin. Illustrative fatty acids may include, but are not limited to, such as palm kernel oil, palm oil, coconut oil, olive oil, laurel oil, or the like, or combinations thereof. Illustrative fatty acids may also include animal fats, such as tallow. Illustrative fatty acids may also include, but are not limited to, fatty acid sources having fatty acid distributions similar or substantially similar to natural or synthetic fatty acid sources (e.g., natural animal fats or oils, natural vegetable fats or oils, individual fatty acids, etc.).

### Skin Care Agents

The personal care composition may include one or more skin care agents. Any suitable skin care agents that do not adversely affect the stability and/or efficacy of the personal care composition may be used. In at least one implementation, the skin care agent may include an emollient configured to maintain a soft, smooth, and pliable appearance to the skin. As is known by those skilled in the art, the emollients may function by remaining on the surface of the skin or in the stratum corneum to act as a lubricant, to reduce flaking, and/or to improve the appearance of the skin.

The skin care agents may generally include one or more polymers (e.g., polyvinylpyrrolidine), protein derivatives (e.g., derivatized hydrolyzed wheat protein), ethoxylated fatty ethers, cellulosics (e.g., hydroxyethylcellulose), or the like, or mixtures and combinations thereof. Illustrative skin care agents may include, but are not limited to, esters comprising an aliphatic alcohol having about 2 to about 18 carbon atoms condensed with an aliphatic or aromatic carboxylic acid including about 8 to about 20 carbon atoms (e.g., isopropyl myristate, decyl oleate, cetearyl isononanate, etc.). The esters may be straight chained or branched. In a preferred implementation, the ester has a molecular weight of less than about 500.

Other skin care agents may include, but are not limited to, polyvinyl-pyrrolidone, polyquaternium-4, polyquaternium-7, polyquaternium-10, guar gum derivatives, hydroxypropylmethylcellulose, hydroxyethylcellulose, a polyethylene glycol, a methyl ether of a polyethylene glycol, quaternium-79, wheat germamidopropyl hydroxypropyl dimonium hydrolyzed wheat protein, stearyl methicone, dimethicone copolyol, dimethicone propyl PG betaine, poly(sodium styrene sulfonate), sorbitan oleate, steareth-2, steareth-21, isoceteth-20, PEG-7 glyceryl cocoate, PEG-75 lanolin, glycereth-26, PPG-5-ceteth-20, a C₁₂-C₂₀ alcohol, canola oil, glyceryl laurate, triglyceryl monostearate, glyceryl monostearate, vitamin E acetate, sunflower seed amidopropylethyldimonium ethylsulfate, sodium PEG-7 olive oil carboxylate, PPG-1 hydroxyethyl caprylamide, PPG-2 hydroxyethyl cocamide, mineral oil, petrolatum, aloe barbadensis, isostearamidopropylmorpholine lactate, strontium acetate, palmitamidopropyltrimonium chloride, or the like, or combinations thereof. In a preferred implementation, the skin care agent is or includes a conditioner, such as a cationic cellulose polymer (e.g., polyquaternium-7).

### Salts

The personal care composition may include one or more salts configured to modify the one or more surfactants, if present, of the personal care composition. For example, the salts may be configured to at least partially modify a cloud point of the surfactants to thereby control the haze or transparency of the personal care composition. The salts may be or include one or more inorganic salts including, but not limited to, sodium sulfate, magnesium sulfate, sodium chloride, sodium citrate, or the like, or combinations thereof. The amount of any one or more of the salts may be at least partially determined by the type and/or amount of the surfactants included in the personal care composition. In at least one implementation, the amount of any one or more of the salts may be about 0.1 weight %, 0.2 weight %, 0.3 weight %, 0.4 weight %, or 0.5 weight % to about 0.6 weight %, 0.7 weight %, 0.8 weight %, 0.9 weight %, or about 1.0 weight %.

### Additional Optional Components/Ingredients

The personal care composition may include one or more additional optional ingredients. Illustrative optional ingredients may include, but are not limited to, one or more dyes, fragrances (e.g., limonene, ethyl butyrate, linalool, and/or oils, such as citronellol, coumarin, benzyl salicylate, etc.), buffers and buffering agents (e.g., inorganic phosphates, sulfates, and carbonates), pH adjusters (e.g., acids and/or bases), preservatives (e.g., parabens, hydantoins, imidazolines, etc.), thickeners, viscosity modifiers, antioxidants (e.g., etidronic acid, etc.), foam enhancers, chelating agents (e.g., EDTA, phosphates, pentasodium pentetate, etidronic acid, etc.), skin conditioning agents, opacifiers, hydric solvents, hydrotropes, antimicrobials (e.g., trichlorocarbanilide (TCC), triclosan, geraniol, carvacrol, citral, eucalyptol, catechol, 4-allylcatechol, hexyl resorcinol, methyl salicylate, etc.), sunscreen actives, anti-aging compounds, essential oils and extracts (e.g., rosewood, jojoba, etc.), polyols, titanium dioxide, abrasives (e.g., particulate matter), acaricidal agents (e.g., benzyl benzoate), or the like, or combinations thereof.

Illustrative antimicrobials may include, but are not limited to, triclocarban, triclosan, or the like, or combinations thereof. Illustrative anti-aging compounds may include, but are not limited to, alpha hydroxy acids, beta hydroxy acids, or the like, or combinations thereof. Illustrative sunscreen actives may include, but are not limited to, butyl methoxy benzoylmethane, or the like, or combinations thereof. Illustrative polyols may include, but are not limited to, glycerol, sorbitol, propylene glycol, polyethylene glycol, or the like, or combinations thereof. Illustrative abrasives or particulate matter may include, but are not limited to, silica, talc, calcium carbonate, polyethylene beads, jojoba beads, lufa, oat flour, or the like, or combinations thereof. Illustrative vitamins may include, but are not limited to, vitamins such as vitamin A, K, or combinations thereof.

Illustrative basic pH adjusters may include, but are not limited to, ammonia; mono-, di-, and tri-alkyl amines; mono-, di-, and tri-alkanolamines; alkali metal and alkaline earth metal hydroxides; and the like, and combinations thereof. For example, the basic pH adjuster may be ammonia, sodium hydroxide, potassium hydroxide, lithium hydroxide, monoethanolamine, triethylamine, isopropanolamine, diethanolamine, triethanolamine, or the like, or combinations thereof.

Illustrative acidic pH adjusters may include mineral acids and polycarboxylic acids. The mineral acids may be or include hydrochloric acid, nitric acid, phosphoric acid, sulfuric acid, or the like, or combinations thereof. The polycarboxylic acids may be or include citric acid, glycolic acid, lactic acid, or the like, or combinations thereof.

The preservatives may be included in the personal care composition in an amount greater than 0.00 weight % and less than or equal to about 3.0 weight % or about 2.0 weight %. Illustrative preservatives may include, but are not limited to, benzalkonium chloride; benzethonium chloride, 5-bromo-5-nitro-1,3-dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynl butyl carbamate, butyl paraben; ethyl paraben; methyl paraben; propyl paraben, mixture of methyl isothiazolinone/methyl-chloroisothiazoline in a 1:3 wt. ratio; mixture of phenoxythanol/butyl paraben/methyl paraben/propylparaben; 2-phenoxyethanol; tris-hydroxyethyl-hexahydrotriaz-ine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadam-antane chloride; sodium benzoate; organic acids, sorbic acid, lactic acid, citric acid, or the like, or combinations thereof.

In an exemplary implementation, the personal care composition is a solid cleaning composition including the carrier and the antioxidant system. The solid cleansing composition may further include water, glycerin, one or more perfumes or fragrances, charcoal, or any combination thereof. In a preferred implementation, the solid cleansing composition includes the carrier or the cleansing component, one or more fragrances, the antioxidant system, and charcoal, and the antioxidant system may include at least a combination of vitamin C and vitamin E. In at least one implementation, the personal care composition may prevent pollution damage to skin, provide anti-pollution benefits to skin, remove pollution damage in and/or on skin, or provide pollution healing properties to skin as compared to the same or a similar personal care composition without the carrier and/or the antioxidant system disclosed herein.

### METHODS

### Method for Preparing a Personal Care Product/Composition

The present disclosure may provide methods for preparing a personal care product or a personal care composition thereof. The method may include mixing, stirring, combining, or otherwise contacting a carrier and an antioxidant system including one or more antioxidants disclosed herein. The carrier may include soap having a mixture or blend of about 85 wt % to about 95 wt % C₁₆-C₁₈ fatty acids and about 5 wt % to about 15 wt % C₁₂-C₁₄ fatty acids, based on the total weight of the soap. The antioxidant system may include one or more of vitamin E, vitamin C, or combinations thereof. In a preferred implementation, the antioxidant system includes a combination of vitamin E and vitamin C. In an even more preferred implementation, the vitamin E includes gamma-tocopherol in an amount relatively greater than any one or more of the other isomers in vitamin E, or the vitamin E only includes the gamma-tocopherol isomer. The antioxidant system or the antioxidants thereof may be present in an effective amount to prevent pollution damage or lipid peroxidation in and/or on skin when applied to the skin prior to exposure to ozone. The antioxidant system or the antioxidants thereof may also be present in an effective amount to reduce, remove, or treat pollution damage or lipid peroxidation in and/or on skin when applied to the skin after exposure to ozone.

### Method for Preventing Pollution Damage to Skin

The present disclosure may also provide methods for preventing pollution damage or lipid peroxidation in and/or on skin. For example, the method may include reducing the rate of lipid peroxidation of skin over time. The method may include contacting any one or more of the personal care compositions disclosed herein with the skin prior to exposing the skin to pollution, such as ozone. For example, the method may include contacting a personal care composition including a carrier and an antioxidant system or the antioxidants thereof with skin. The antioxidant system or the antioxidants thereof may be present in an effective amount to prevent pollution damage or lipid peroxidation in and/or on skin when applied to the skin prior to exposure to ozone.

### Method for Reducing or Treating Pollution Damage to Skin

The present disclosure may also provide methods for reducing, removing, or otherwise treating pollution damage or lipid peroxidation in and/or on skin. The method may include contacting any one or more of the personal care compositions disclosed herein with the skin after exposing the skin to pollution, such as ozone. For example, the method may include contacting a personal care composition including a carrier and an antioxidant system or the antioxidants thereof with skin after exposure to ozone. The antioxidant system or the antioxidants thereof may be present in an effective amount to treat, remove, or otherwise reduce pollution damage or lipid peroxidation in and/or on skin when applied to the skin exposed to pollution.

Method for Cleaning Skin and Concurrently Preventing and/or Treating Pollution Damage to Skin

The present disclosure may also provide methods for cleansing skin while concurrently preventing and/or treating pollution damage or lipid peroxidation in and/or on skin. The method may include contacting skin with an effective amount of a personal care composition including a carrier and an antioxidant system or the antioxidants thereof. The antioxidant system or the antioxidants thereof may be present in an effective amount to treat, remove, or otherwise reduce and/or treat pollution damage or lipid peroxidation in and/or on skin when applied to the skin.

### Personal Care Product/Composition for Use in Preventing Pollution Damage to Skin

The present disclosure may also provide a personal care composition for use in preventing pollution damage or lipid peroxidation in and/or on skin. The personal care composition may include a carrier and an antioxidant system or the antioxidants thereof.

### Personal Care Product/Composition for Use in Treating Pollution Damage to Skin

The present disclosure may also provide a personal care composition for use in treating pollution damage or lipid peroxidation in and/or on skin. The personal care composition may include a carrier and an antioxidant system or the antioxidants thereof.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

A base bar soap composition or control (1) was prepared by combining soap chips containing C₁₆-C₁₈ fatty acids and C₁₂-C₁₄ fatty acids, charcoal, fragrance, and excipients, according to Table 1. Specifically, soap chips including about 92.5% C₁₆-C₁₈ fatty acids from tallow and about 7.5% C₁₂-C₁₄ fatty acids from lauric oils, charcoal, fragrance, and excipients, were combined with one another according to Table 1. A test bar soap composition (2) was then prepared by adding varying amounts of vitamin E and vitamin C, according to Table 2. The vitamin E included Tocopherols, particularly, gamma-Tocopherol (γ-Tocopherol). As indicated in Table 2, the control did not include any amount of either vitamin E or vitamin C. As such, it should be appreciated that any differences between the control (1) and test (2) bar soap compositions may be attributed to the respective combination of the vitamin E and vitamin C.

TABLE 1
Composition of Base Bar Soap Composition

| | Amount |
|---|---|
| Ingredient/Component | (wt %) |
| Soap chips containing 92.5% C₁₆-C₁₈ | 97.78 |
| fatty acid and 7.5% C₁₂-C₁₄ fatty acids | |
| Charcoal | 0.09 |
| Fragrance | 2.1 |
| Excipients | Balance |

TABLE 2
Compositions of Control and Test
Bar Soap Compositions (1) and (2)

| | Base Bar Soap | Vitamin E | Vitamin C |
|---|---|---|---|
| # | Composition (wt %) | (wt %) | (wt %) |
| (1) Control | 100.0 | 0.0 | 0.0 |
| (2) | 99.96 | 0.03 | 0.01 |

### Example 2

The test (2) bar soap composition was evaluated for its efficacy in delivering γ-Tocopherol to skin utilizing the base bar soap composition, particularly, the soap chips containing C₁₆-C₁₈ fatty acids and C₁₂-C₁₄ fatty acids, as a vehicle. Porcine back skin was used as skin models for the control (1) and test (2) bar soap composition. Particularly, 5 cm×10 cm skin model samples were prepared from the same piece of the porcine back skin, and each of the control (1) and test (2) bar soap composition was used to treat a respective skin model sample.

To evaluate deposition or delivery of γ-Tocopherol to the skin, water with a flowrate of about 6 liters/min and at a temperature of about 37° C. was used. Both the skin model sample and the bar soap composition was wetted with water for about five seconds. Then, the bar soap composition was contacted or rubbed onto the skin for about 15 seconds and lathered for an additional 15 seconds. After rubbing and lathering, the skin model sample was allowed to rest for about 45 seconds and subsequently rinsed for about 15 seconds. Excess water was then shaken off of the skin model sample and the skin model sample was allowed to air dry for about 5 minutes.

To quantify the amount of γ-Tocopherol delivered to each of the skin model samples, a cup scrub method was utilized to extract the γ-Tocopherol from the respective skin model samples. Particularly, a glass cup having a diameter of about 1.5 cm was placed in the center of the skin model sample, 3 mL of methanol was disposed inside the cup and rubbed with a glass rod for about 30 seconds. The methanol was then removed with a pipette and disposed in a 10 mL volumetric flask. This process of extraction was repeated or performed in duplicate, thereby resulting in a total of about 6 mL of methanol. The volumetric flask was then filled with additional methanol to provide a final volume of about 10 mL.

Each of the methanol samples extracted from the respective skin model samples was then evaluated with a HPLC spectrophotometer with a UV detector. The HPLC spectrophotometer was utilized in the following configuration: a mobile phase of 99/1 methanol/water, a C18 reverse phase column commercially available from Agilent Technologies, Inc. of Santa Clara, CA, a flow rate of about 1 mL/min, an injection volume of about 80 μL, and a detection wavelength of about 298 nm.

The calculated concentration of γ-Tocopherol was then corrected for dilution and total reaction volume. The amount of γ-Tocopherol extracted from each of the skin model samples tested with the respective control (1) or test (2) bar soap composition is summarized in Table 3.

TABLE 3
Amount of γ-Tocopherol Extracted from Skin Models Treated
with Control (1) and Test (2) Bar Soap Compositions

| | Amount of γ-Tocopherol |
|---|---|
| # | (μg/cm²) |
| (1) Control | Not detected |
| (2) | 0.019 ± 0.006 |

As illustrated in Table 3, the test (2) bar soap composition effectively delivered γ-Tocopherol to skin.

### Example 3

The efficacy of the test bar soap composition (2) of Example 1 for preventing pollution damage to skin or its ability to provide anti-pollution benefits to skin was evaluated and compared to the control (1) bar soap composition of Example 1.

To evaluate the efficacy of the control (1) and test (2) bar soap compositions for preventing pollution damage to skin or their ability to provide anti-pollution benefits to skin, lipid peroxidation of skin was evaluated. Particularly, the amount of lipid peroxidation generated in skin exposed to ozone after treatment with either the control (1) and test (2) bar soap compositions was evaluated. The amount of lipid peroxidation present in and/or on the skin was measured with a lipid peroxidation assay kit, which monitored the presence of a biomarker for lipid peroxidation; specifically, the presence of malondialdehyde (MDA), a byproduct of lipid peroxidation. Pig skins were utilized as skin models for each of the control (1) and test (2) bar soap compositions. Particularly, two pieces of 4×2 inch defrosted pig skins were cut into four 2×2 inch samples. Each of the control (1) and test (2) bar soap compositions was tested on both of the defrosted pig skins. Particularly, the control (1) bar soap composition was tested on a 2×2 piece from the first pig skin and a 2×2 piece from the second pig skin. Similarly, the test (2) bar soap composition was tested on a 2×2 piece from the first pig skin and a 2×2 piece from the second pig skin.

To evaluate the control (1) and test (2) bar soap composition, each of the pig skin samples was pre-wetted with running water (100 mL/sec at a temperature of about 37.7° C.) for about 2 to 3 seconds, contacted or rubbed with either the control (1) or the test (2) bar soap composition for about 15 seconds, then lathered with a gloved finger for about 15 seconds. After pre-wetting, washing, and lathering, the pig skin samples were allowed to rest for an additional period of about 60 seconds before rinsing with the tap water for about 15 seconds.

After rinsing, each of the pig skin samples was exposed to ozone. Particularly, each of the pig skin samples was placed in a CH-1 ozone chamber (Model 106-L) obtained from Oxidation Technologies, LLC. of Inwood, IA, and exposed to about 100 ppm ozone for about 30 minutes. After exposing each of the pig skin samples to ozone, MDA was extracted from each of the pig skin samples. The MDA was then extracted from each of the pig skin samples via a cup scrub method. Particularly, a glass cup was placed in the center of the pig skin samples, 500 μL of ethanol was disposed inside the cup and rubbed with a glass rod for about 1 min. The ethanol was then removed and disposed in a centrifuge tube. The process of extraction was performed in duplicate, thereby resulting in a total of about 1 mL of ethanol. The extracted samples were then centrifuged for about 1 minute at about 10,000 RPM.

To quantify the amount of MDA, a calibration curve was first prepared. Particularly, a 20 μM standard solution of MDA was diluted with ethanol to varying concentrations. A 25 ml solution of thiobarbituric acid (TBA) was prepared by mixing one container of TBA powder (provided in the kit) with about 7.5 mL of acetic acid, and about 17.5 mL of deionized (DI) water. 200 μL of each of the respective MDA dilutions was then contacted with about 600 μL of the TBA solution, incubated at about 95° C. for about 60 min, and cooled in an icebox for about 10 min. 200 μL of the mixture was then pipetted into a well plate in duplicate. Each sample was then measured using a SPECTRAMAX M5, obtained from Molecular Devices, LLC. of San Jose, CA. Specifically, each of the samples was excited at a wavelength of about 532 nm and emission was measured at a wavelength of about 553 nm. The measured emission intensity of each of the samples and the corresponding concentration of the dilute MDA solution were then utilized to prepare the calibration curve.

To quantify the amount of MDA extracted from each of the pig skin samples, about 200 μL of the centrifuged MDA samples obtained from the cup scrub method was contacted with about 600 μL of the TBA solution, incubated at about 95° C. for about 60 min, and cooled in an icebox for about 10 min. Similar to the samples prepared for the calibration curve, about 200 μL of the mixture was then pipetted into a well plate in duplicate. Each sample was then measured using a SPECTRAMAX M5. Specifically, each of the samples was excited at a wavelength of about 532 nm and emission was measured at a wavelength of about 553 nm. The measured emission intensity of each of the samples was then utilized to determine the amount of MDA in each of the samples extracted from the pig skins using the calibration curve. The amount of MDA measured from pig skins exposed to ozone before treatment with the control (1) and test (2) bar soap compositions is summarized in Table 4. Each of the control (1) and test (2) bar soap compositions was tested in duplicate generating 4 samples (n=4). An adjusted mean and ±2 standard error, which corresponds to a 95% confidence interval on the mean was utilized for the data analysis via 2-way ANOVA.

TABLE 4
MDA Measured from Pig Skin Treated with Control (1) and
Test (2) Bar Soap Compositions Before Exposure to Ozone

| | MDA Average* | |
|---|---|---|
| SAMPLE | (μM) | Std Dev |
| Control (1) | 3.97 | 0.17 |
| Test (2) | 3.36 | 0.17 |

*P < 0.004

As illustrated in Table 4, relatively less MDA was measured in the pig skins treated with the test bar soap composition (2) than the pig skins treated with the control bar soap composition (1). Specifically, a decrease in MDA of about 15% was observed between the control bar soap composition (1) and the test bar soap composition (2). MDA is a byproduct for lipid peroxidation, which is a marker for pollution. As such, it should be appreciated that the test bar soap composition (2) including the combination of including the combination of vitamin E (γ-Tocopherol) and vitamin C surprisingly and unexpectedly prevented pollution damage to skin when exposing the skin to ozone.

### Example 4

The efficacy of the control (1) and test (2) bar soap compositions prepared in Example 1 for removing pollution damage in and/or on skin or its ability to provide pollution healing properties to skin was evaluated. Pig skins were utilized as skin models for each of the control (1) and test (2) bar soap compositions. Particularly, two pieces of 4×2 inch defrosted pig skins were cut into four 2×2 inch samples. Each of the control (1) and test (2) bar soap compositions was tested on both of the defrosted pig skins. Particularly, the control (1) bar soap composition was tested on a 2×2 piece from the first pig skin and a 2×2 piece from the second pig skin. Similarly, the test (2) bar soap composition was tested on a 2×2 piece from the first pig skin and a 2×2 piece from the second pig skin.

Each of the pig skin samples was placed in a CH-1 ozone chamber (Model 106-L) and exposed to about 100 ppm ozone for about one hour, which resulted in the generation or production of MDA in and/or on the pig skin samples. After exposure to ozone, the four 2×2-inch pig skin samples were pre-wetted, washed, and lathered with the control (1) or test (2) bar soap composition. Tap water with a flow rate of about 100 mL/sec at a temperature of about 37.7° C. was utilized for the pre-wetting, washing, and lathering. The general procedure included pre-wetting each of the pig skin samples with the running water for about 2 to 3 seconds, contacting or rubbing either the control (1) or the test (2) bar soap composition with the respective pig skin sample for about 15 seconds, then lathering with a gloved finger for about 15 seconds. After pre-wetting, washing, and lathering, the pig skin samples were allowed to rest for an additional period of about 60 seconds before rinsing with the tap water for about 15 seconds.

The MDA was then extracted from each of the pig skin samples via a cup scrub method, similar to Example 3. Particularly, a glass cup was placed in the center of the pig skin samples, 500 μL of ethanol was disposed inside the cup and rubbed with a glass rod for about 1 min. The ethanol was then removed and disposed in a centrifuge tube. The process of extraction was performed in duplicate, thereby resulting in a total of about 1 mL of ethanol. The extracted samples were then centrifuged for about 1 minute at about 10,000 RPM. The amount of MDA from each of the samples was measured similar to Example 3 and is summarized in Table 5.

TABLE 5
MDA Measured from Pig Skin Treated with Control (1) and
Test (2) Bar Soap Compositions After Exposure to Ozone

| | MDA Average* | |
|---|---|---|
| SAMPLE | (μM) | Std Dev |
| Control (1) | 4.82 | 0.12 |
| Test (2) | 4.02 | 0.12 |

*P < 0.0005

As illustrated in Table 4, relatively less MDA was measured in the pig skins treated with the test bar soap composition (2) than the pig skins treated with the control bar soap composition (1). Specifically, a decrease in MDA of about 17% was observed between the control bar soap composition (1) and the test bar soap composition (2). MDA is a byproduct for lipid peroxidation, which is a marker for pollution. As such, it should be appreciated that the test personal care composition (2) including the combination of vitamin E (γ-Tocopherol) and vitamin C surprisingly and unexpectedly reduced, removed, or otherwise treated pollution damage in skin exposed to ozone.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

What is claimed is:

1. A personal care composition comprising a carrier and an antioxidant system comprising one or more antioxidants, wherein the antioxidant system is present in an effective amount to treat, inhibit, or prevent lipid peroxidation of skin.

2. The personal care composition of claim 1, wherein the one or more antioxidants comprise selenium, carotenoids, lycopene, lutein, zeaxanthin, one or more vitamins, or combinations thereof.

3. The personal care composition of claim 1, wherein the one or more antioxidants comprise one or more vitamins.

4. The personal care composition of claim 3, wherein the one or more vitamins comprise vitamin C, vitamin E, or combinations thereof.

5. The personal care composition of claim 2, wherein the one or more vitamins comprise a combination of vitamin C and vitamin E.

6. The personal care composition of claim 4, wherein the vitamin E comprises one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof.

7. The personal care composition of claim 4, wherein the vitamin E comprises gamma-tocopherol.

8. The personal care composition of claim 4, wherein the vitamin C comprises ascorbic acid, derivatives thereof, or combinations thereof.

9. The personal care composition of claim 8, wherein the derivatives of ascorbic acid comprise one or more of L-ascorbic acid, calcium ascorbate, calcium l-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof.

10. The personal care composition of claim 8, wherein the derivatives of ascorbic acid comprise sodium ascorbyl phosphate.

11. The personal care composition of claim 10, wherein the vitamin E comprises gamma-tocopherol.

12. The personal care composition of claim 1, wherein the carrier comprises a cleansing component, and wherein the cleansing component comprises a soap.

13. The personal care composition of claim 12, wherein the soap comprises a fatty acid soap.

14. The personal care composition of claim 12, wherein the soap comprises C₁₆-C₁₈ fatty acids and C₁₂-C₁₄ fatty acids.

15. The personal care composition of claim 14, wherein the C₁₆-C₁₈ fatty acids are present in an amount of from about 85 wt % to about 95 wt %, and wherein the C₁₂-C₁₄ fatty acids are present in an amount of from about 5 wt % to about 15 wt %, based on a total weight of the soap.

16. The personal care composition of claim 14, wherein the C₁₆-C₁₈ fatty acids are present in an amount of about 92.5 wt %, and wherein the C₁₂-C₁₄ fatty acids are present in an amount of about 7.5 wt %, based on a total weight of the soap.

17. The personal care composition of claim 1, wherein the personal care composition is a solid personal care composition.

18. A method for preparing the personal care composition of claim 1 for treating or preventing lipid peroxidation of skin, the method comprising contacting the carrier and the antioxidant system with one another.

19. A method for preventing lipid peroxidation of skin, the method comprising:
contacting the personal care composition of claim 1 with the skin; and
exposing the skin to pollution,
wherein the personal care composition prevents lipid peroxidation of the skin.

20. A method for treating lipid peroxidation of skin, the method comprising contacting the personal care composition of claim 1 with the skin, wherein contacting the personal care composition with the skin reduces lipid peroxidation in and on the skin.
